(19) United States
(12) Patent Application Publication
(10) Pub. No.: US 2022/0280083 A1
(43) Pub. Date: Sep. 8, 2022

(54) SYSTEM AND METHOD FOR GENERATING AND PROVIDING EMOTIONAL WELLNESS SOLUTIONS

(71) Applicant: HappinessFactors Inc., San Jose, CA (US)

(72) Inventor: Anita Kumari, San Jose, CA (US)

(21) Appl. No.: 17/194,166

(22) Filed: Mar. 5, 2021

(51) Int. Cl. A61B 5/16; G16H 20/30; G06T 13/40

(52) U.S. Cl. CPC A61B 5/165; G16H 20/30; G06T 13/40

(57) ABSTRACT

Improved computerized techniques are provided with which the user, via an innovatively configured user interface, interacts with a computerized, practical application, the one or more processors of which are configured to guide the user in a way that improves their wellness.

### BACKGROUND OF THE INVENTION

### Technical Field

This invention relates generally to the field of computerized wellness applications. More specifically, this invention relates to a computer processor measuring wellness-related factors, processing such factors, subsequently generating a wellness-related solution, and presenting such wellness-related solution on a graphical user interface.

### Description of the Related Art

People face various mental and emotional health issues on an everyday basis in today's world. Examples include trauma, accident, unexpected calamities, loss of a loved one or a job, financial crisis, stress, burnout, post-traumatic stress disorder (PTSD), bad relationships, anxiety, worry, inability to cope with everyday life, chronic illnesses, postpartum depression, personal and professional struggles after childbirth, just not feeling good enough or any other negative life experience/occurrence. It is known that there are major stressors affecting wellness and that people rarely take the time to check their emotional state.

A system and method for assessing global wellness and developing a maintenance/treatment program is taught in U.S. Patent Application 20170199189, SYSTEM FOR ASSESSING GLOBAL WELLNESS (filed Jun. 26, 2015) to J. Wade. Specifically, this technique involves compiling a plurality of data sources, including, (a) a subject's medical, dental and family histories, and (b) a body fluid sample (e.g., an oral swab or saliva) for detecting and determining levels of one or more biomarkers, wherein these data are used to assess the subject's global health and wellness landscape, which is then compared to a reference/ideal wellness landscape, and the comparison used to design a maintenance therapy/treatment plan.

However, such prior art techniques require invasive technologies, such as those requiring data from invasive mechanisms (e.g., a body fluid sample) and require privacy technologies such as requiring private data from medical records (e.g., a subject's medical, dental and family histories) for assessing the subject's global health and wellness landscape. These and other prior art techniques require the subject to complete wellness examination forms and require dental diagnostic evaluations. Such prior art technologies are technologically archaic and methodologically archaic (e.g., lack capability to provide variety in the field of wellness).

### SUMMARY

Improved computerized techniques are provided with which the user, via an innovatively configured user interface, interacts with a computerized, practical application, the one or more processors of which are configured to guide the user in a way that improves their wellness.

### BRIEF DESCRIPTION OF THE DRAWINGS

FIG. 1 is a flow diagram for the process of generating a wellness solution in response to user input, in accordance with an embodiment;

FIG. 2 is a schematic diagram of a system for generating and providing emotional wellness/mental health solutions, in accordance with an embodiment;

FIGS. 3A-3Y are screenshots of a sample application illustrating a sequence of screens, in accordance with an embodiment; and

FIG. 4A-D is a flow diagram showing an example of a method for generating a wellness solution in response to user input, in accordance with an embodiment; and

FIG. 5 is a block schematic diagram of a system in the exemplary form of a computer system according to an embodiment.

### DETAILED DESCRIPTION

Improved computerized techniques are provided with which the user, via an innovatively configured user interface, interacts with a computerized, practical application, the one or more processors of which are configured to guide the user in a way that improves their wellness.

As a preliminary matter, it should be appreciated that the innovation or embodiments may each be referred to herein as a platform, system, method, practical application, application, or app.

The innovation recognizes that the conscious mind takes up about 10% of a person's mind versus the subconscious mind using about 90% of the person's mind. Thus, the computer-implemented platform provides holistic emotional wellness solutions.

In an embodiment, emotional wellness encompasses mental health and a holistic integration of physical, mental, and spiritual well-being, fueling the body, engaging the mind, and nurturing the spirit. Emotional wellness is the ability to successfully handle life and its stressors, build healthy, effective relationships, adapt to change, have the tools to not just survive, but thrive beyond the difficult/traumatic happenings by taking and engaging with the learnings to get better, not bitter. Emotional wellness helps build resilience and positivity. People who are emotionally well have fewer negative emotions, and show signs of positivity and resilience.

In an embodiment, emotional wellness encompasses and helps balance the various dimensions of wellness including:
Physical,
Emotional/Mental,
Intellectual,
Social,
Spiritual,
Environmental,
Financial/Occupational and others.

In an embodiment, emotional wellness is the ability to feel and express human emotions and make decisions by effectively integrating feelings, emotions, thought processes, values. Emotional wellness inspires stress reduction, self-care, relaxation and developing inner strength. It helps develop assertiveness, conflict resolution and confrontation skills, create healthy self-esteem, develop and maintain intimate and loving relationships. It helps an individual become the best version of their self. How one feels can affect their ability to carry out their daily activities, their relationships, their overall mental health, and how they live their life.

Technical areas integrated into the platform include, but are not limited to: apps, gaming, Artificial Intelligence (AI), Neuro Linguistic Programming (NLP), Mental Emotional Release (MER), Hypnotherapy, Psychology, Yoga, Ayurveda, Upanishads and Vedic Teachings and Values, Sutras, Puranas, Meditation, Sanatan Dharm, Shlokas, Energy Healing and other holistic methodologies. In a nutshell, the innovation combines the best of the East and the West to offer total holistic wellness solutions with focus on the subconscious mind which is the foundation for 90% of behavioral patterns. The system and method make providing the solutions more interesting and comprehensive.

For background, it should be appreciated that while there are some app-based solutions in the market such as Headspace, Calm, Happify, Aura, and Insight Timer, they mostly focus on a small niche, meditation and primarily target the conscious mind.

The innovation solves the following problems, as follows. It has been found that one in five Americans suffer from some kind of mental or emotional wellness issue. Most available solutions are meditation centric or offer symptom relief instead of targeting the root cause, each of which may create more issues, and most insurances only cover limited number of visits. Other options available in the market are complicated, distributed, and expensive. The innovative platform offers an emotional wellness ecosystem that is configured to provide various solutions available in one place. Whether someone needs to feel better, happier, less anxious, feel loved, have better relationships or just needs to feel more confident, different levels of solutions that can be customizable are implemented for every need. In an embodiment, the system and method include any combination of an app, coaching, webinars, seminars, trainings/workshops, books and more. The platform also offers access to therapists, coaches and other holistic practitioners.

Overview of An Embodiment of the App Process:

In an embodiment, the app processes the following steps, as shown in Table A. It should be appreciated that the order of steps may vary, depending on the embodiment.

```
TABLE A
  1) Cover Screen with logo, forest background, register/create login, agree to disclaimer, privacy policies.
  2) Dashboard screen
  3) Happiness-X Land screen
  4) Jungle screen where a cute puppy is looking for something - behind stones, trees, flowers, asks monkey and/or others, sits down in a forlorn way when he can’t find it. Suddenly a rainbow and/or clouds pops up with butterflies flying around it, flowers erupting and different topics
flash: happiness, rapport, productivity, motivation, moodiness, anxiety,
depression, worthlessness, not good enough, energy, enthusiasm etc.
There’s voice in the background that’s sings, what can I help you with today? How can I make you happy today? And/or something similar
The puppy excitedly gets up and starts dancing while the song in the background says “thank you for helping me today, I am looking for happiness today”. And/or something similar
  5) Q & A Screen where the user is asked a few questions They can pick an answer from the drop-down menu and also put some details in the text box below. Questions may include what’s their definition of happiness, what’s making them happy? What’s making them unhappy?
  6) Rating screen where they rate themselves on a scale of 1 to 10 and how three of their closest peers rate them on a scale of 1 to 10, etc.  7) Next screen is the score (average of all 3)
  8) They get the score and explanation of the score
  9) Solution screen: includes balloon game, video, quiz, and activity
10) They check a box after they finish and get accolades. For example,
an accolade is a clapping animation with overlaid or juxtaposed text
saying, “Fantastic, what a score!” or “Congrats, way to go!” or “Wow,
you did it!” and so on.
11) They are then taken to Happiness-X Land to get updated score & change it by rocket/parachute game.
11) Happiness X land is where they get their score. They get accolades
based on current score-prior score. They have the option to go back and
retake the quiz to improve their score, as well as the game score(s) or other improvement scores. They also get the percentile compared to
other users who took the same.
12) They have the option to go to the website to get more tools/services, refer folks, give feedback.
```

Examples of uses of the innovative platform, according to an embodiment, are as follows. The system and method can be used for holistic treatment of emotional/mental ailments, to have a better quality of relationships and life, to feel fulfilled, to be a better leader, team member, be more compassionate, kind and community oriented, to achieve goals and get more successful in life, to live a high energy, healthy, meaningful life, to have enough mental muscle, resilience, self-control and clarity to avoid bad habits and addictions, to get more career and personal success and have better relationships, to have more peace, relaxation collaboration and faith, to get more prosperity and abundance, and to have better alignment between mind, body and spirit and thus be better equipped to effectively handle the curve balls of life. In an embodiment, the innovative emotional wellness ecosystem platform is a one stop shop for unique holistic solutions to lead a happy, healthy, fulfilling, and peaceful life.

An overview of a similar embodiment is described as follows. After the user creates their profile and logs in, they are asked to choose the emotional wellness area they would like to work on. For example, the user interface (UI) screen may present a screen with the words, “Choose the emotional wellness module you want to work on” and/or something similar. The user is asked their definition, what makes them feel that emotion? What makes them feel opposite of that emotion? Or they are asked something to that effect. In the embodiment, the next screen may ask the user to rate themselves on how they feel (e.g., on a scale from 1-10). In the embodiment, the application obtains, from a predetermined number (e.g., three) of presumably close friends/colleagues/other-related individuals or entities, ratings on how such friends would rate the user. For example, such close friends/colleagues/other-related individuals or entities may be presented, e.g., via a user interface on their mobile devices, with a list of available ratings, such as 1-10, and an explanation to the ratings (range based). In the embodiment, a UI is presented to the user, in which a score, or other type of measurement, is provided. Such score is the computed average of the scores obtained from the close friends/colleagues/other-related individuals or entities. In an embodiment, such average score is presented as a balloon game score. That is, the embodiment improves over prior art techniques by innovatively initiating a gaming module and combining the computed score with the gaming module to facilitate gameplay in a wellness application. Further, the balloon gameplay may be selected based on demographic information of the user. For example, a person under the age of 25 is adept at gameplay and is used to more instant gratification than their older counterparts. Thus, the balloon game score may optimize the user's retention in engaging with the application than simply presenting a score according to prior art techniques. Further, the innovation requests that the user input things which make them happy. For example, the system or method may present a text box to the user with a prompt saying, “Please list the top 5 things which may you happy.” Similarly, the innovation requests that the user input things which make them unhappy. For example, the system or method may present a text box to the user with a prompt saying, “Please list the top 5 things which may you unhappy.” In accordance with the embodiment, the next screen takes the user through a solution that consists of a video, a Neuro Linguistic Programming (NLP) quiz, and an activity. Then, the user is taken to the game screen. From watching the video, taking part in the NLP quiz, and/or partaking in the activity, the innovative platform processes the new data from such quiz and/or activity and computes an adjust or new score. The system presents the adjusted or new score to the user. Depending on the score, which may reflect whether the user's wellness or happiness has improved during the live session, the system may also provide the option for more products (e.g., more products related to happiness or the user's wellbeing) and direct the user to the website from the present application. In an embodiment, the application may ask for feedback from the user, preferably online, such as via a text box, etc.

In an embodiment, the innovation includes a cloud server that is communicably connected to an Emotional Wellness application on a client computing device of a user (e.g., a platform) that provides holistic solutions to mental wellness combining the Eastern techniques from Vedas, Meditation, Ayurveda, Yoga, Energy and Spirituality with Western techniques of Psychology, Neuro Linguistic Programming, MER, Hypnotherapy and more.

The innovation provides solutions for emotional wellness/mental health issues including but not limited to stress relief, productivity, self-esteem, focus, anxiety, moodiness, unexpected loss, depression, trauma, relaxation, productivity, anxiety, worry, anger, helplessness, not feeling enough, energy, happiness, building rapport, effective relationships, chronic illnesses, age/memory related issues, body image issues, personal & professional struggles after child birth, calamities or any other life occurrence/experience.

The innovation is configured to be used by corporations and individuals to get emotionally well and thrive in life.

Prior art techniques focus on a small niche with an instruction type process while the innovation improves upon technological advances by integrating a gamified approach as well as targeting the subconscious mind and holistic solutions. The innovative platform is configured to address of the root causes of emotional wellness issues and configured to provide long lasting solutions without requiring drugs, alcohol or marijuana.

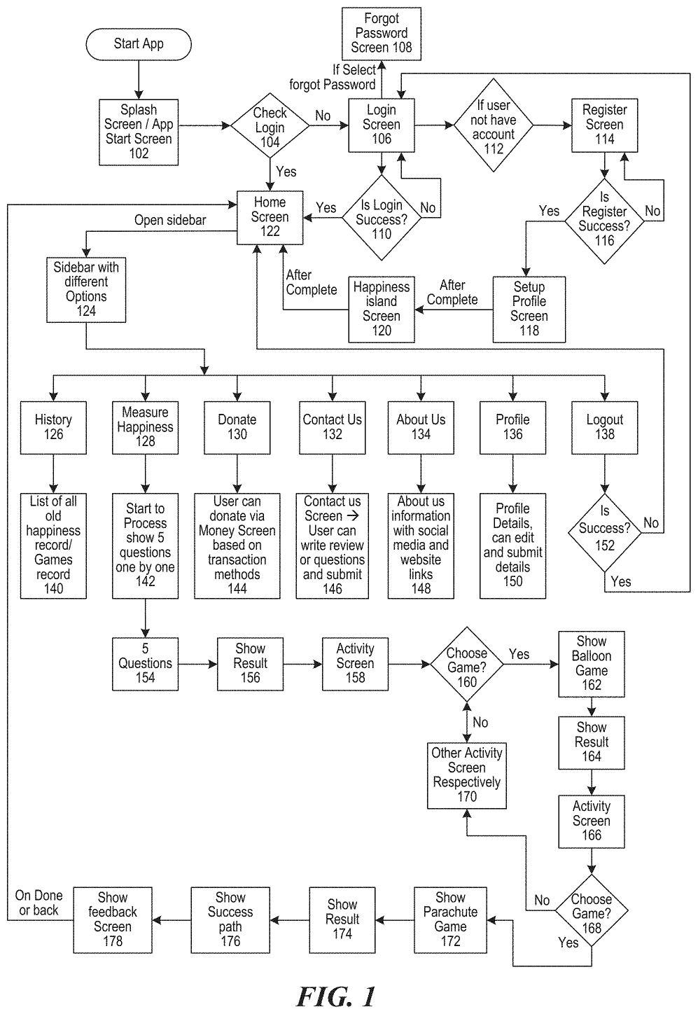

An embodiment can be understood with reference to FIG. 1, a flow diagram for the process of generating a wellness solution in response to user input. Upon starting up the application on their client device (e.g., a smartphone, a personal computer, a computerized tablet), the platform presents a start screen of the application at step 102. At step 104, the system checks the login credentials of the user. If the system determines that the credentials are valid, at step 122 the application presents the home screen of the application, the further processing of which is discussed below.

If the system determines that the credentials are not valid, at step 106 the application presents a login screen, through which the user may enter login data. For example, if the user forgot their password, the user is presented with a forgot password screen at step 108. In an embodiment, after receiving the required information, from user input, in such screen, the process proceeds to perform activities related to receiving user input indicating that the user forgot their password. The innovation may use current techniques for rectifying a forgotten password, e.g., sending a temporary password to a registered email address, that are not taught herein. If the system determined, at step 110, that the login was successful, then command goes to step 122, and the system displays the home screen. If, at step 110, the login was not successful, command returns to step 106, described above. Based on user input from step 106, the system may determine that the user does not have an account, at step 112. In response to the indication, such as a signal or specific input, that the user does not have an account, the application may present a register screen at step 114, through which to obtain user input about the new user account. At step 116, the system determines if the required registration is valid, i.e., if the registration is successful. If not, the application returns to step 114. If yes, the application presents a setup profile screen at step 118. Once sufficient data is received by the system from the setup profile screen at step 118, the system presents a happiness island screen at step 120.

It should be appreciated that the happiness island screen is by way of example only and the system may be configured to display other types of screens that serve the same purpose, i.e., to try to trigger an inner emotion or state of happiness. Further, in an embodiment, the innovative placement of such happiness-related screen at this point in the sequence of displayed screens may be considered a unique element that is integrated into the practical application of generating wellness solutions based on user input.

After the happiness island screen is displayed at step 120, the system returns to the home screen at step 122. In an embodiment, the system may keep the happiness screen open for a predetermined length of time, e.g., 7 seconds, and after such predetermined length of time may display the home screen. In another embodiment, the system may display the same happiness-related screen until receiving user input to change the screen. In another embodiment, the system may display two or more happiness-related screens, an optionally rotate through such screens, until a predetermined end time or until receiving user input to leave such screens.

At step 124, the application presents a plurality of options, each option indicating a sub-program, module, or component with which the user may engage. In an embodiment, the options may be presented as a sidebar. Such sidebar may be present on the home screen, linked to via the home screen, or accessed by other means of the art.

In an embodiment, the plurality of different options includes one or more of history, user definition of happiness, what percentile the user falls under, measure of happiness, donate, contact us, about us, profile, logout, and get directed to the company website to view more services/products. At step 126, the history option was selected via user input and, in response, the system displays a list of prior happiness records and prior games records, as per step 140. At step 128, the measure happiness option was selected via user input and, in response, the system initiates an innovative measure happiness process, as indicated in step 142 and as described in detail below. At step 130, the donate option was selected via user input and, in response, the system displays a money screen based on transaction methods through which the system may obtain user input related to a donation, as per step 144. At step 132, the contact us option was selected via user input and, in response, the system displays a contact us screen, as per step 146. Via such screen, the system obtains user input data about user reviews or questions, submitted by such user. At step 134, the about us option was selected via user input and, in response, the system displays about us information with social media and website links, as per step 148. At step 136, the profile option was selected via user input and, in response, the system displays profile details which are editable and may be submitted by the user, as per step 150. At step 138, the logout option was selected via user input and, in response, the system determines whether the logout was successful, as per step 152. If no, then the system returns to step 122, the home screen. If yes, then the system returns to the login screen at step 106.

Responsive to the system receiving the signal that the measure happiness 128 process has been selected based on user input, and the system initiating the innovative measure happiness process, as indicated in step 142, the system presents, via the user interface, a predetermined number of questions or inquiries, also referred to as a quiz, to the user, as indicated in step 154, as discussed below in more detail. It should be appreciated that the measure happiness process is by way of example and is not meant to be limiting. Other indicators of an individual's wellness may be considered by the innovation. For example, the process at 128 may be a sub-component or application for a user to improve their leadership skills via the configured platform's generated solutions. Other non-exhaustive examples of wellness-related modules that the platform may be configured to provide solutions for are any one or more of: mental health issues, Attention Deficit Hyperactivity Disorder (ADHD) and other attention and focus related issues; the autism spectrum; ageing, memory and age-related issues/illnesses such as forgetfulness, dementia, etc., chronic illnesses, self-esteem and confidence issues, interpersonal relationship issues, stress, and other wellness issues.

Examples of five questions at step 154 in accordance with an embodiment may include, but are not limited to, “What is your definition of happiness?”; “What makes you happy?”; “What makes you unhappy?”; “How do you rate your happiness now?”; and “How do three of your closest peers/friends rate your happiness?”

In an embodiment, the algorithm determines the number of questions and which questions based on specific criteria.

For example, the specific criteria include data from a user's profile and the selection of the measure happiness topic may be in the form of a request that contains user identification information. The platform uses the user identification information to retrieve a user profile for that user. The user profile indicates specific identifying information about the user, such as for example, the demographic of the user, the number of times the user has executed the measure happiness component of the application. The platform can determine whether a user is novel and has never participated in the measure happiness component before or is at a certain level of happiness as a result of playing the measure happiness component before.

In an embodiment, the platform is configured for the administrator to add questions or tags (e.g., success, happiness, etc.) in the administration panel and the same is made visible to the user based on the specific criteria pertaining to the user. When the user submits answers, criteria is extracted from the answers (e.g., feeling happy or not feeling successful) and submitted to the library of solutions. The library of solutions is configured to check whether the criteria has been fulfilled. Once the criteria are matched, then the library transmits, for delivery to the user, the solutions and the scores (described below). The code is written to ascertain that the right answer is provided to the user by the library and the other answers in the background are kept in a separate bin, e.g., for future purposes. The criteria to match the user's request or answers with the solutions provided are predetermined. In an embodiment, extracting criteria from the answers is performed using available software or code that parses text input and identifies therefrom known or acceptable words and/or phrases.

In an embodiment, the user input process incorporates steps (e.g., five) that not only includes the user's ratings, but also the ratings of three (or another predetermined number) of their closest peers. It has been found that three is an optimal number. These ratings provide an optimal, e.g., 360 degree, view into the user's emotional state at the time of using the application or platform. When the user answers what's making him/her unhappy at that time, their answer selection determines the solution they are offered later by the platform. For example, if the user says they are unhappy because they are stressed, the platform determines and provides them the solution related to stress. Whereas, if a user provides an answer that they are unhappy because they are tired, then the system determines and provides them the solutions related to being tired. The backend code makes certain that the solutions relevant to the user are shown and all other solutions are kept in a separate library (bin).

In an embodiment, tags are used in place of questions. For example, the tags can represent the emotional state of the user, such as success or happiness. Thus, the platform is configured to transmit and present a multitude of tags for presentation to a user and the user can select the one or more tags that represent their emotional state. The selection of the tags is transmitted to the server and used as input into the algorithm that provides the one or more solutions to the user.

In an embodiment, the system is configured to generate and present to the user specific and predetermined questions, such as the following set of questions (or similar) depicted in Table B, for obtaining required data with which to process and determine a level of happiness of the user.

TABLE B

| 1) | What am I happy about in my life right now? | |
|---|---|---|
| 2) | What about that makes me happy? | |
| 3) | In a day, what percentage of the time I feel happy? | |
| 4) | What percentage am I unhappy? | |
| 5) | Please rate in order of priority how the following impact your | |
| | happiness | |
| | i) | Money |
| | ii) | Relationships |
| | iii) | Career |
| | iv) | Health |
| | v) | Success |
| | vi) | Lifestyle |
| | vii) | Home |
| | viii) | Exercise/workout |
| | ix) | Education/learning |
| | x) | Vacation |

| | |
|---|---|
| 6) | Do you practice gratitude daily? |
| 7) | Do you complain daily? |
| 8) 9) | Are you competitive or collaborative? How well do you take criticism? |
| 10) | Do you celebrate successes for your loved ones? |

It should be appreciated that other embodiments may be configured with similar questions to present or be configured to pose a different number of questions to the user. For example, in a similar leadership application, it may be determined that it is sufficient for the platform to be configured to present three or more questions to the user, the data from which may be used to generate and present an online customized solution for improving the user's leadership skills.

At step 156, after receiving the user input from the presented questions, the system processes the input data in accordance with an innovative algorithm, discussed in detail below, for generating and showing the result. In an embodiment, system, based on the generated result, the system generates an activity screen and presents such screen to the user at step 158. For example, the system may present one type of activity screen to one user, based on that user's results, and a different activity screen to a different user, based on that different user's different results. In an embodiment, the activity screen is interactive so that, at step 160, the system can receive a user input signal indicating which game the user selects to play. It should be appreciated that such activity and games are innovatively configured to correlate to the application, e.g., measure happiness. For example, the activity screen may be designed specifically to induce feelings of happiness. As well, the games made available on the system may also be innovatively configured to resonate with the user's feelings of happiness, as derived through consultation with experts such as doctors, psychologists, therapists, psychiatrists, etc. In an embodiment, examples of activities users would engage in are guided meditation, state change activity such as brief walk, dance, talk to a peer/friend, etc. In other applications, the activities and the games may be configured, with input from expert entities, to correlate with the desired outcome, e.g., improved leadership skills.

At step 162, the system received user input that a specific game was selected at step 160, e.g., a balloon game. In an embodiment, such game is innovatively configured to address or target a specific human attribute. For example, in an embodiment, a balloon game is provided, with which the user can quickly, actively, and possibly somewhat mindlessly click on balloons. More specifically, it has been found that millennials like to click quickly on items on a screen, as a means to release stress. Thus, the innovation provides the result of the answered questions of step 154 as a set of pop-able balloons.

In an embodiment, each balloon is color coded to a particular emotion and when the user clicks on the balloon, such balloon falls into the Happy or Unhappy buckets based on the color coding of the balloon.

A concrete example is discussed below. In an embodiment, a happiness score is generated for the user, based on the input answers to the questions. For example, if a user's happiness is rated 2 out of 10, then the system is configured to present a set of 10 balloons, 2 of which may be colored in a standard joyful color, such as yellow or orange, and the system may present the remaining balloons in colors that have been previously determined to represent sad feelings, such as black, grey, or brown. In the embodiment, such colored balloons are presented to the user. The user clicks on each balloon and each balloon animatedly falls into one of two appropriate GUI boxes, e.g., labeled “happy” and “unhappy”, respectively. That is, the innovation is configured so that the user can obtain their score while simultaneously releasing stress, by clicking the balloons, which causes the balloons to drop into the appropriate bin. As mentioned, it should be appreciated that other types of games, which require online interaction and which simultaneously cause a satisfactory human behavior result, may be contemplated.

At step 164, the system presents the result of the game, e.g., the user's score as indicated by the number of balloons that had fallen in each bin, as discussed above.

At step 166, either after a predetermined amount of time, e.g., ten seconds, or upon user input, the system presents a second activity screen. It should be appreciated that other embodiments may be configured to present a multitude of activity screens at more and/or different strategic places within the flow of the application. For example, based on peer-accepted studies of human behavior, the system may be configured to present an activity screen after every other screen display. In another example, a set of two or more activity screens may be presented contiguously, one after the other. In an embodiment, the activity screen is configured to improve the user's happiness level state, e.g., by showing a nature-related background or animated cute animals and the like.

At step 168, the system requests input from the user as to whether the user selects to play a game. If not, the system is configured to present another or different activity screen, as shown in step 170. In an embodiment, the system is configured to present a quiz as the activity or along with the activity to the user. The system has been innovatively configured to choose the quiz based on the previously system-determined score as in step 164. The innovative platform is configured to process the further input data from the quiz to generate a new, and hopefully improved, happiness score for the user. An exemplary quiz is presented below, in Table C. It should be appreciated that such quiz is by way of example and is not meant to be limiting:

TABLE C
Emotional Wellness Quiz

```
Do you know your emotional wellness quotient? Take the following quiz and find out.
Score details:
Excellent = 5, Very Good = 4, Good = 3, Fair = 2, Poor = 1
  1) How would you rate your ability to develop and maintain
relationships? ______
  2) How well do you accept your mistakes? ______  3) Do you like constructive criticism? ______  4) Are you able to laugh at life? ______  5) How good do you feel about yourself? ______  6) How would you rate your self-love? ______  7) You get an unexpected challenge, how do feel about it? ______  8) Do you have your life under control? ______  9) How well do you handle stress? ______ 10) Are you grounded? ______ 11) How is your energy level? ______ 12) Do you take time regularly to unwind? ______ 13) Do you have control over your life? ______ 14) Do you set priorities regularly? ______ 15) Do you achieve goals regularly? ______ 16) Are you well aware of your strengths? ______ 17) Are you well aware of your weaknesses? ______ 18) Do you feel good about your future? ______ 19) How good are you at expressing your feelings? ______ 20) Do you look forward to the future? ______
Ratings:
20-40: You need to put serious efforts to get emotionally well, before it derails your life. 41-60: You need to make efforts to get emotionally well. 61-80: You have average emotional wellness and need some work. 81-100: You are on the right path. With little effort, you can achieve your dream life.
```

From step 170, the system receives the new data from the user, processes such data, and returns the user to the step 160, presents the option for the user to choose a game.

In an embodiment, after the initial score and explanation, the system presents a quiz to the user, as well as some video learning and activity. The game choice comes after that. Then, the process proceeds, as shown, to step 162, at which the same game is played, but with updated, new results. For example, after having performed an activity as suggested previously, in activity screen 166, such as to take a walk or stretch in place, it is assumed that the user may feel better, e.g., happier, and the user input to the quiz reflects the improved emotional state of the user. Thus, the process repeats in showing the balloon game (for example) with updated codified balloons in step 162.

At step 172, the system receives user input that a game was not chosen. Thus, at this step the system presents a different, conclusive-type game, e.g., the rocket-parachute game. In an embodiment, the rocket-parachute game is based on the life incidences of the user. The user clicks on life experiences. The game is configured such that each positive occurrence (e.g., selection), such as a promotion, getting engaged, and so on, is processed by the game's processor and shows, e.g., in an animation, a GUI of the user being taken up in rocket and each negative experience, such as a job loss, a break up, and so on, is processed by the game's processor to show that the GUI of the user is brought down (e.g., from up in the sky to the ground) in a parachute.

At step 174, the system displays the session's resulting emotional state, as depicted by the rocket-parachute game. For example, the system may determine, based on processing the data and comparing the score to a predetermined threshold, to display the screen that depicts the rocket appearing to shoot up, thus indicating a good level of happiness or emotional state of the user. Continuing with the example, the system may determine, based on processing the data and comparing the score to a predetermined threshold, to display the screen that depicts the rocket ejecting a parachute that falls gently down to the ground, thus indicating a low level of happiness or emotional state of the user. The user is given a predetermined amount of time to play the rocket-parachute game.

At step 176, the system is configured to generate and present a proposed success path to the user. The success path is based on the progress of the user from the time they started the app to the changes in their emotional state till the end of the game. The system also provides suggestions to the user on how to reach their utmost success, e.g., a predetermined amount such as 90th to 100th percentile or 90 to 100% of their level of happiness/emotional state. For example, suppose a user scored 3 in the beginning of the app. After going through the video, activity, quiz and game, suppose his/her new score is 7. The change of the score (4) shows the success in achieving progress towards their desired level of happiness/emotional wellness as well as the effectiveness of the process.

At step 178, after presenting the success path to the user, the system is configured to present a feedback screen to obtain user input data that may be used to ascertain the effectiveness as well as other measurements of the application and customizing the app to the needs of the user and improving it.

Upon receiving some or no user feedback (e.g., receiving a “Done” user-input command), the system returns to step 122, presenting the home screen. From there the user may continue to use the application to ascertain and/or improve their measure of happiness (or whatever aspect they are working to improve, such as leadership skills) or stop.

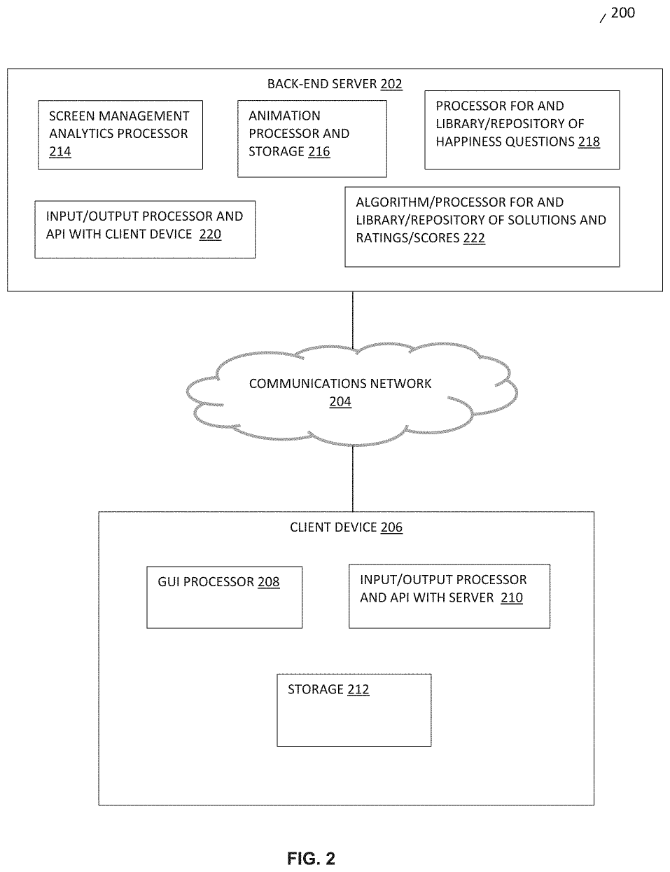

An embodiment of a system architecture can be understood with reference to FIG. 2, a schematic diagram of a system for generating and providing emotional wellness solutions 200. Typically, an individual, such as person wishing to improve their emotional well-being, may open up the application by using their client device 206, for example as described in FIG. 1. The application may be local, for example, previously downloaded onto the client device 206 or the user may access the application at the client device 206 via a web application from a specific website. The client device 206 may store application-related data locally on a local storage (not shown). Thus, references to the client device herein may include local storage accessible by the application. The client device 206 communicates with a back-end server 202 (e.g., cloud) through a communications network, component, or module 204. Exemplary communication networks include wireless communication networks, local area networks (LAN) such as a Wi-Fi network or Ethernet, and/or wide area networks (WAN), such as the Internet, etc. In addition, communications component 204 may represent one or more wired or wireless direct connections. Direct connections may include e.g., Bluetooth, Universal Serial Bus (USB), high-definition multimedia interfaces (HDMI), and custom serial interfaces. In an embodiment, the back-end server 202 hosts one or more processors and databases for processing and storage capabilities. Further, back-end server 202 contains a communications capabilities for communicating with the client device 206 via communications network 204.

In an embodiment, client device 206 contains a GUI processor 208 configured to display the animation, games, activities, and other graphical-user-interface related elements pertaining to the application. GUI processor 208 is configured to display GUI elements from a local application or at a website. Client device 206 contains an input/output processor with API 210 for communicating with the server. For the example, input/output processor and API 210 is configured to transmit ratings to the server and receive solutions from the server. Client device 206 contains a storage 212 that is configured to store data or instructions pertinent to the platform. For example, questions transmitted from the server can be stored in this storage of the client device. Storage 212 can represent a persistent storage or a temporary storage.

In an embodiment, back-end server 202 contains a screen management analytics processor 214 that is configured to generate and transmit to the client device 206 or present on a website a home screen, a rating screen, a solutions screen, and an accolades screen. Further, the screen management analytics processor 214 that is configured to, in association with the solutions screen, generate and transmit a game, a video, a quiz, and an activity. Back-end server 202 contains an animation processor and storage component 216 configured for generating and storing animated images and processes for presentation or delivery to the client device 206. Back-end server 202 contains a processor for and library/repository of happiness questions 218. Processor for and library of happiness questions 218 is configured to determine the appropriate happiness questions to provide for a particular user at the client device 206. Back-end server 202 contains an input/output processor and API with client device component 220. Component 220 is configured to be the interface to the client device 206, via the communications network 204. Back-end server 202 contains an algorithm/processor for and library/repository of solutions and ratings/scores component 222. Component 222 is configured to receive answers and ratings and find matching solutions and explanations thereto.

In an embodiment, the platform is configured to be secure and efficient. For example, the platform employs customized encryption for sensitive data and also uses a unique token for each user.

In an embodiment, the back-end server 202 may use one or more modules that are each communicably connected. For example, in an embodiment, the back-end server 202 has: a present questions module for presenting questions to the user, receiving user input data related to the questions, process such data, and store such data in a database or datastore; a show results module for displaying intermediary results to the user via the application; an activity screen module for determining which elements, such as videos, activities, and quizzes, to present to the user, based on previously received user input and the processing of such input; a choose game module for determining a customized game (e.g., a customized set of balloons or a height of the rocket in the rocket game) to generate and present to the user; a success path module for generating and presenting a customized success path for the user; and a feedback module for receiving user-provided input for post-processing and/or storage. In an embodiment, such different modules are communicably connected by programmed routines that are configured to easily fetch the relevant data from the database with speed and without lag.

In an embodiment, the back-end modules at the back-end server 202 communicate with the front-end modules on the client device 206 through Rest APIs. In an embodiment, a customized framework is built and used at the back-end server 202. The corresponding code is written to ensure that queries are executed with fast speed. The platform's efficient use of APIs makes the system efficient enough To not get overloaded and stuck.

In an embodiment, data may be stored in the cloud (e.g., back-end server 202) or locally (e.g., on client device 206). Typically, most of the data are stored in the cloud. However, on the client device 206, the application side, after login, user data may be stored locally as well. Data is stored at the application side so that the APIs aren't required to be called every time.

In an embodiment, the system provides the user with options such as mantra, guided, or instrumental meditation and other holistic solutions. The system is configured to provide such options to the user as follows. The system uses some key elements or points to obtain user data and determine or find out the user's situation by providing suggestions such as happy, unhappy etc. Such key points are editable at the backend side. The platform obtains data from the application through APIs and store such data in the database through queries. The platform uses unique codes in the framework to make the system fast and secure.

In an embodiment, the platform is configured with unique code to ensure efficiency and high bandwidth in the server, thereby ensuring that the application will not crash if many users (e.g., hundreds of thousands) use the application at the same time.

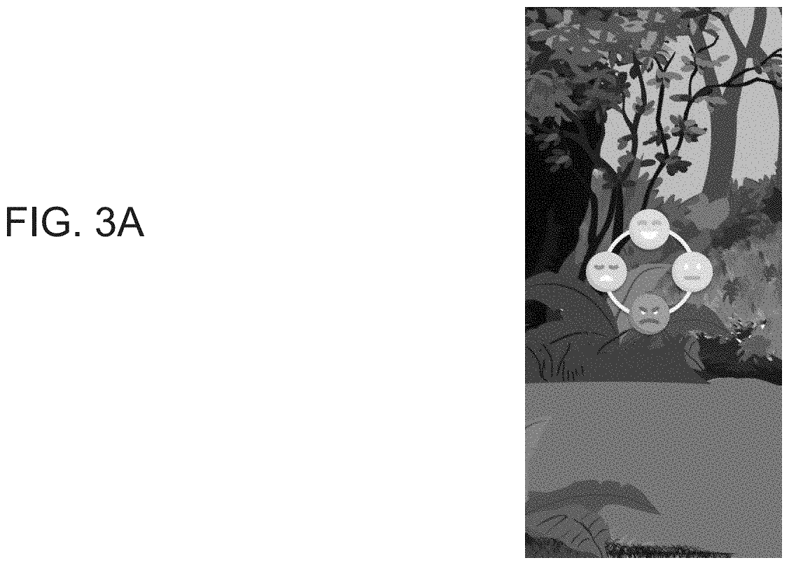

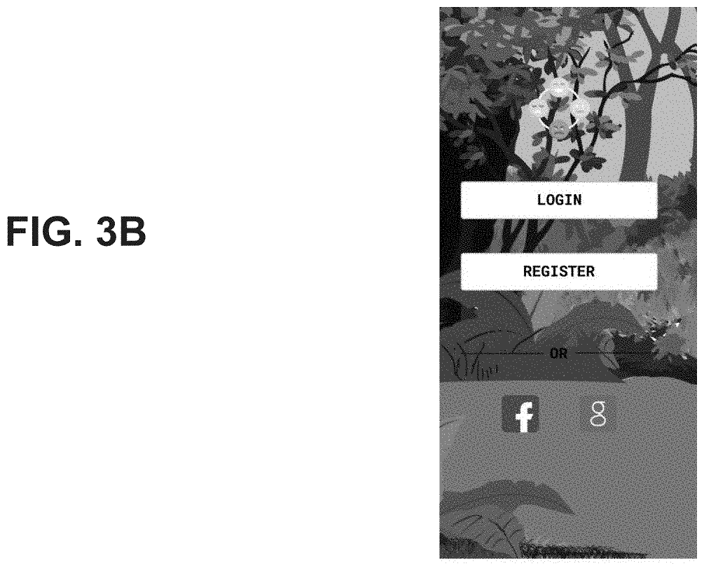

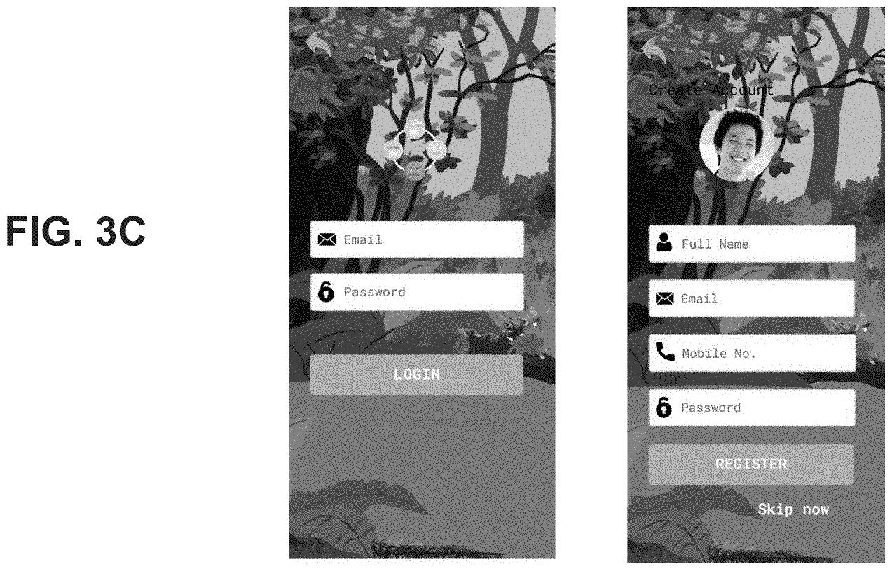

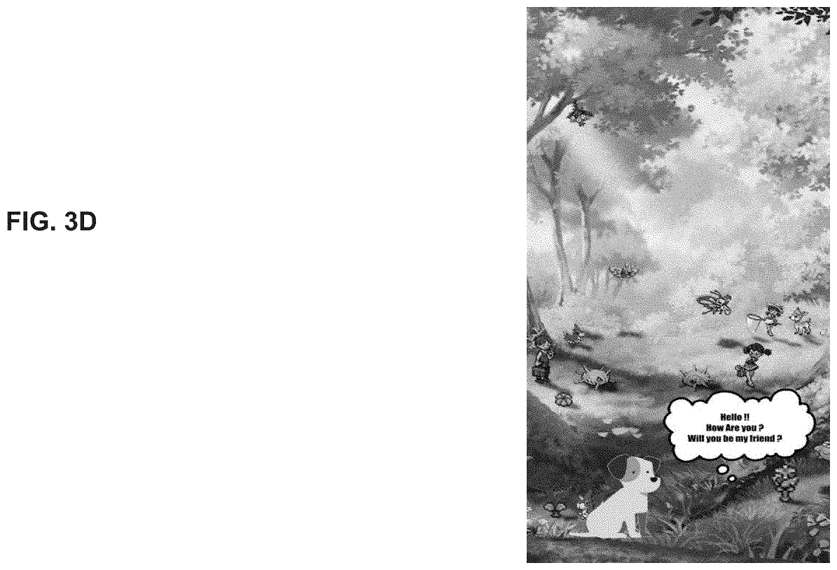

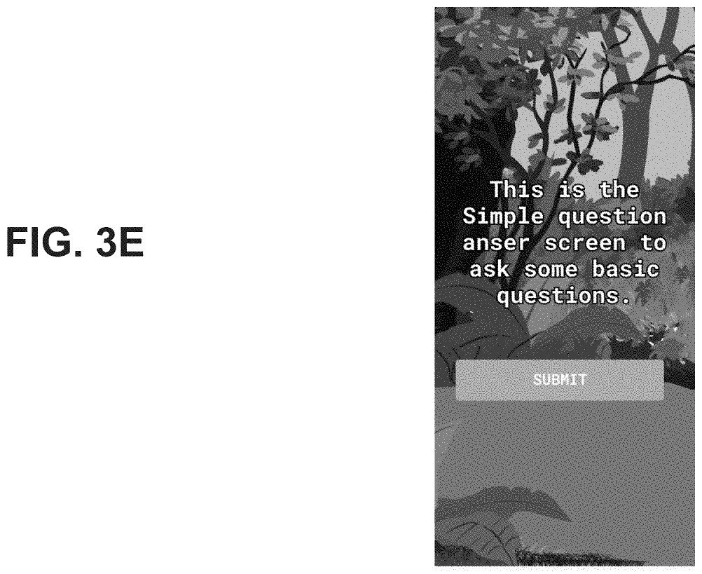

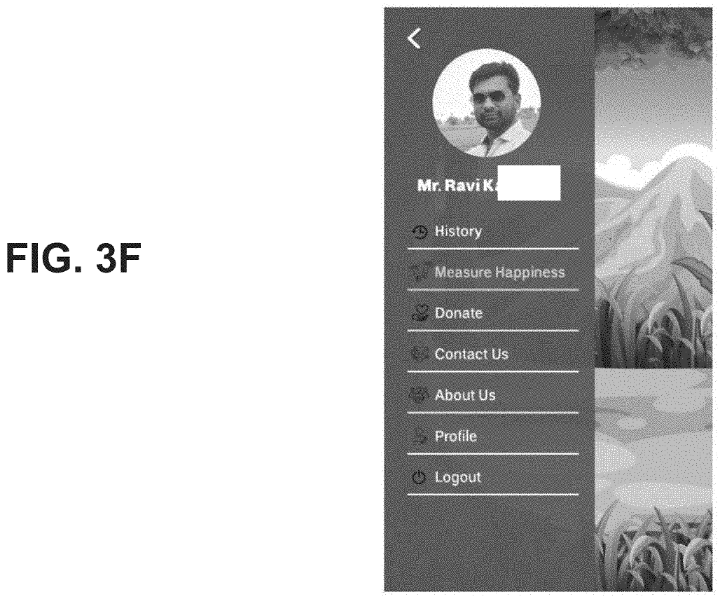

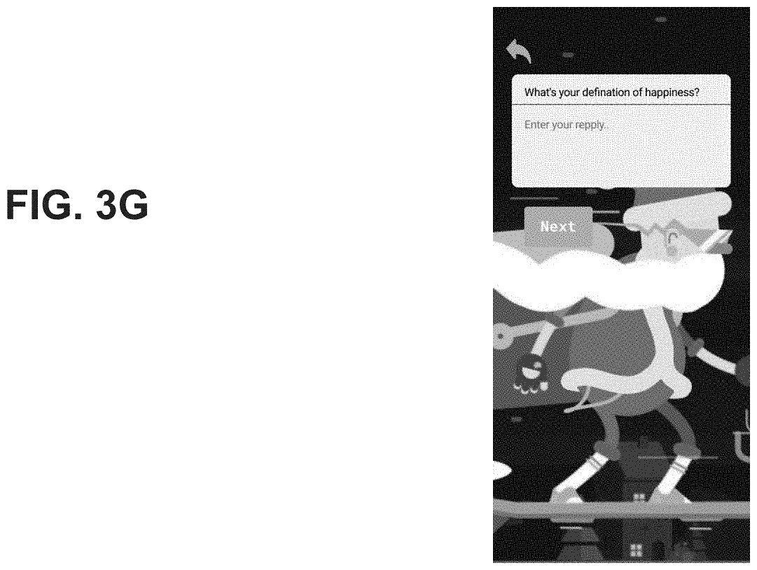

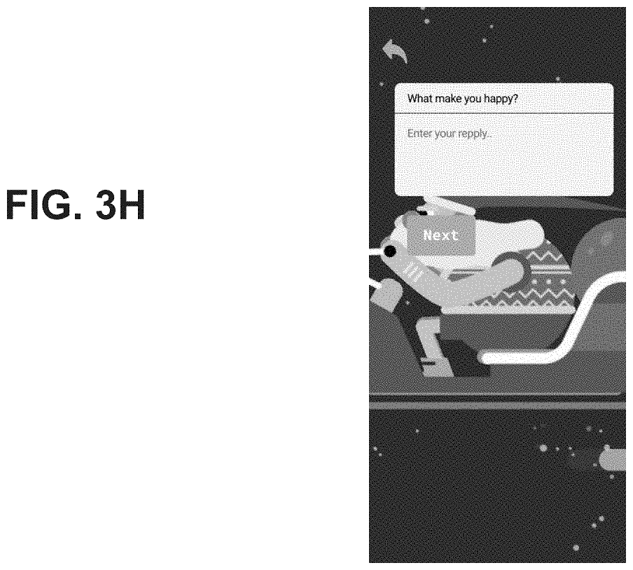

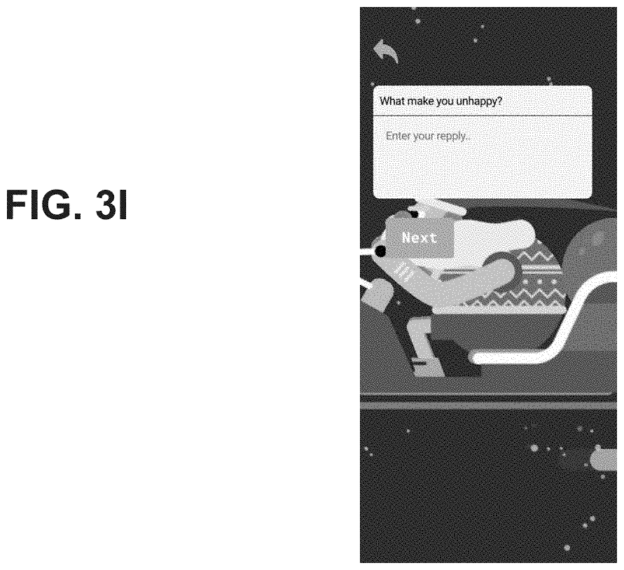

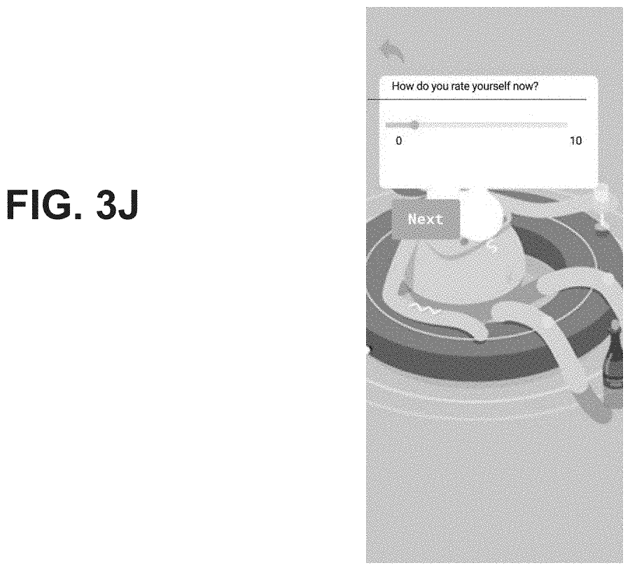

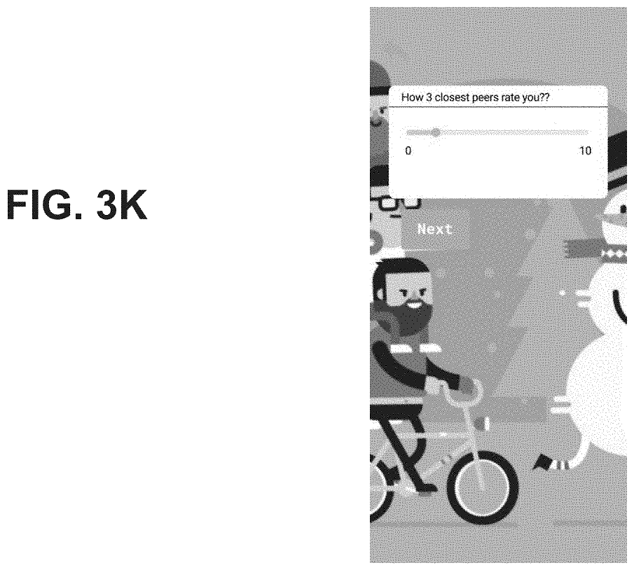

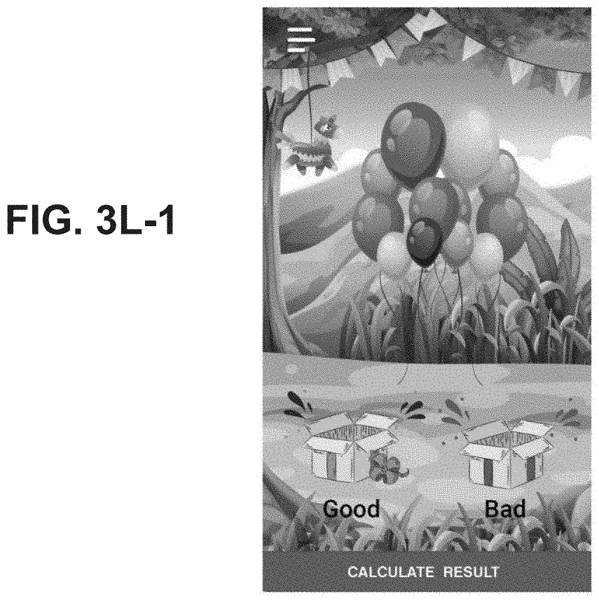

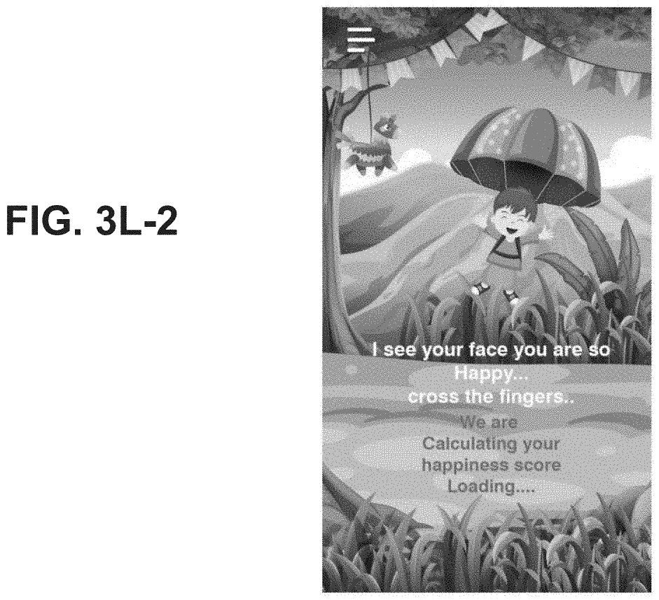

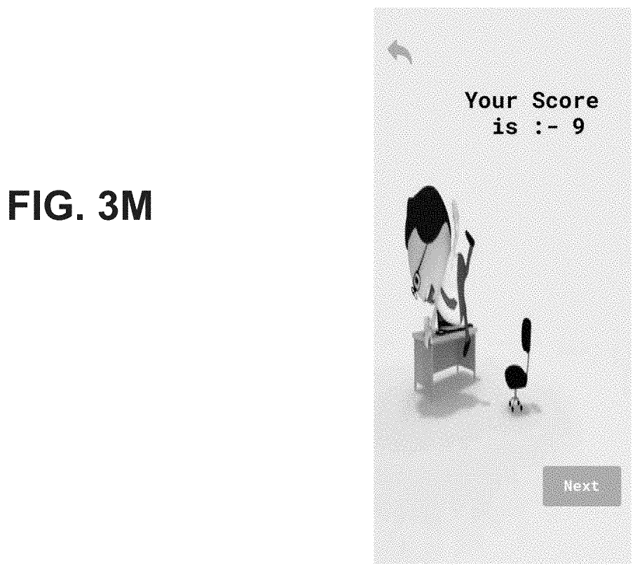

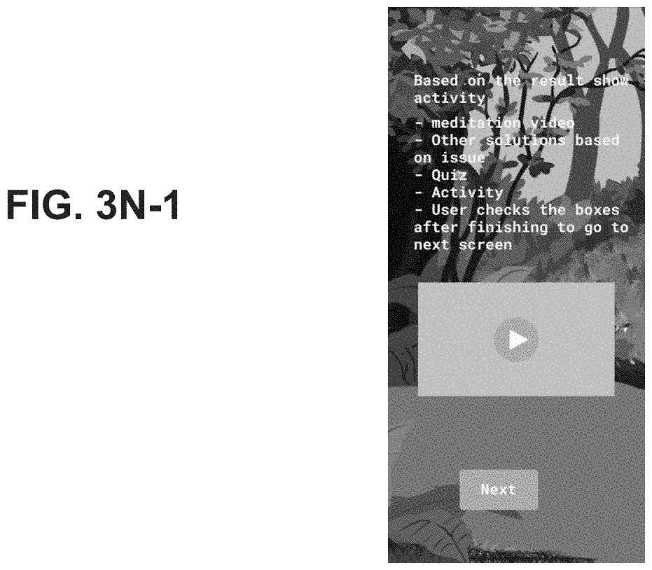

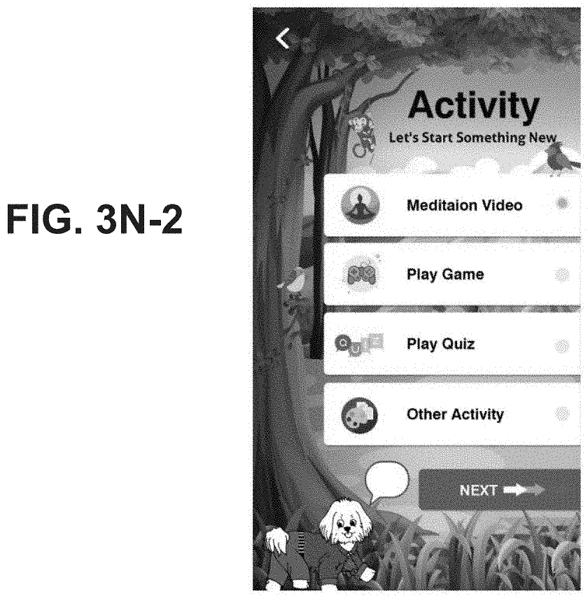

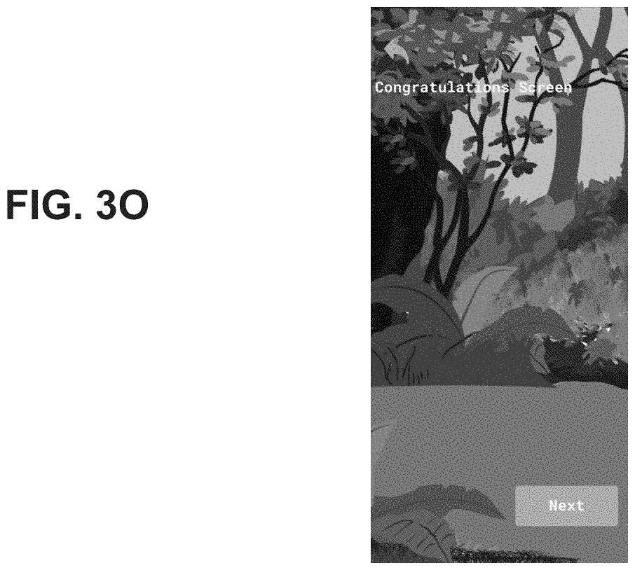

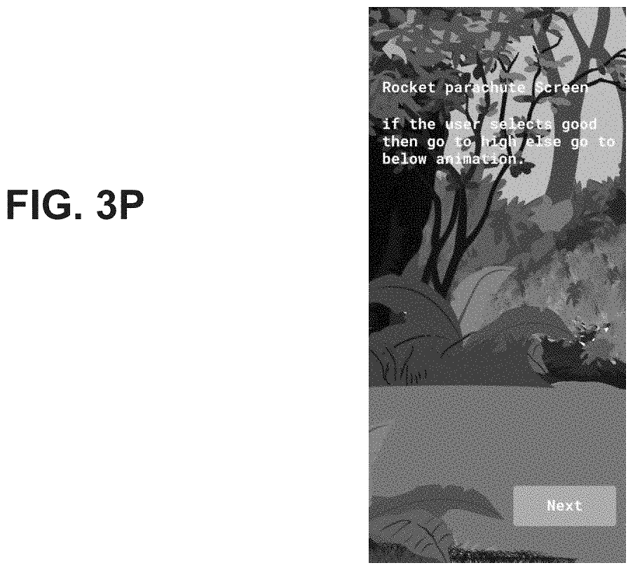

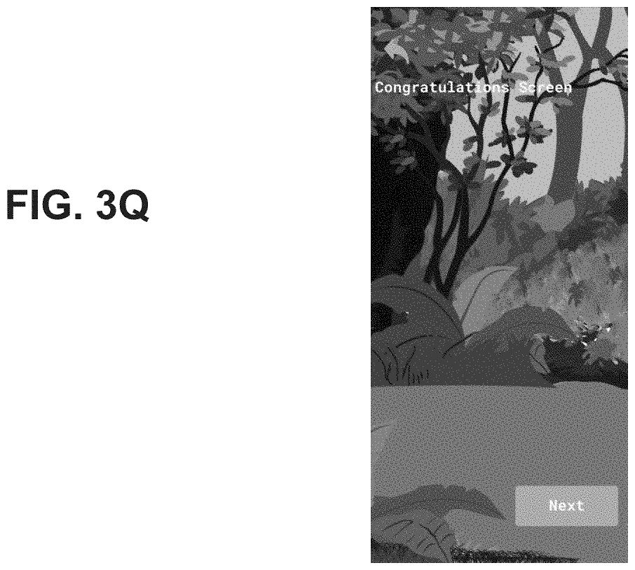

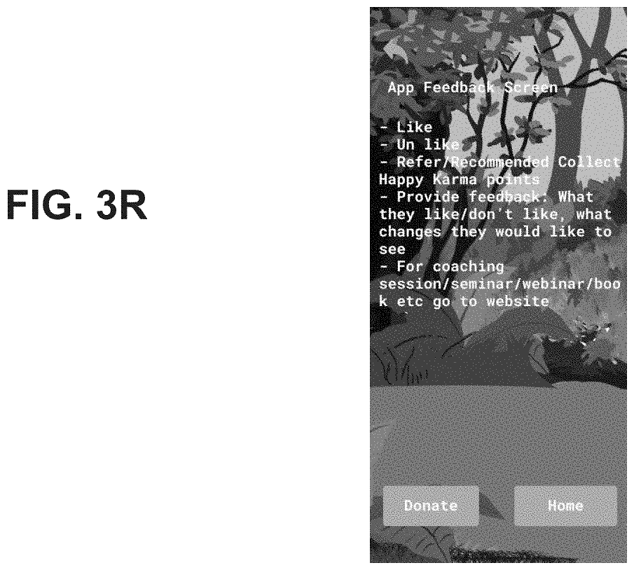

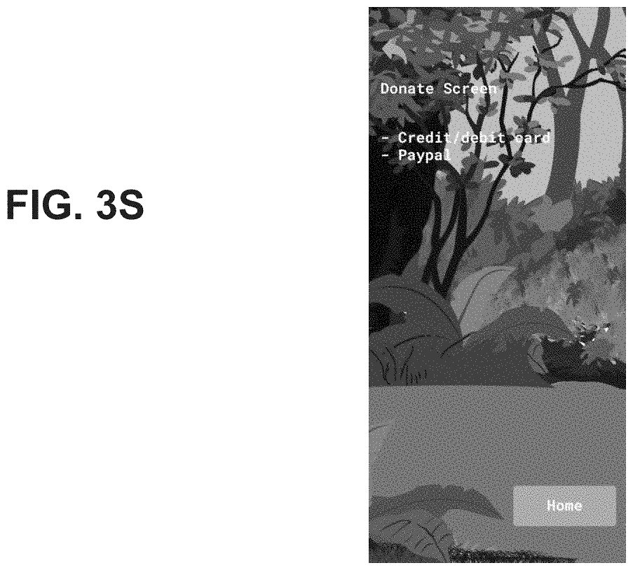

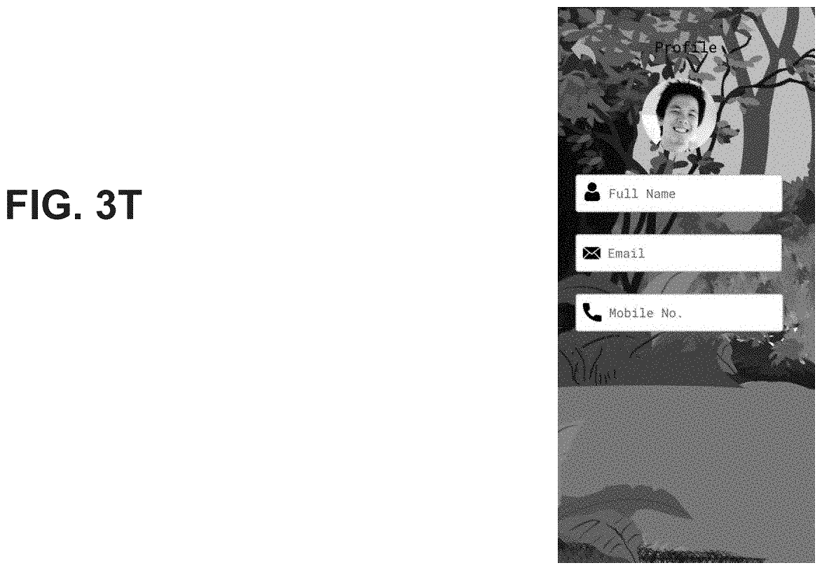

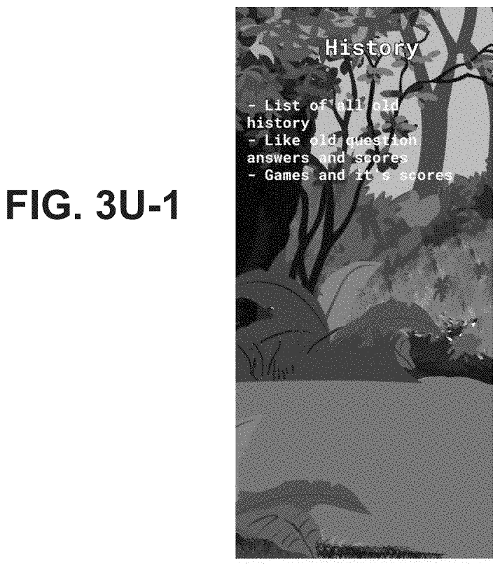

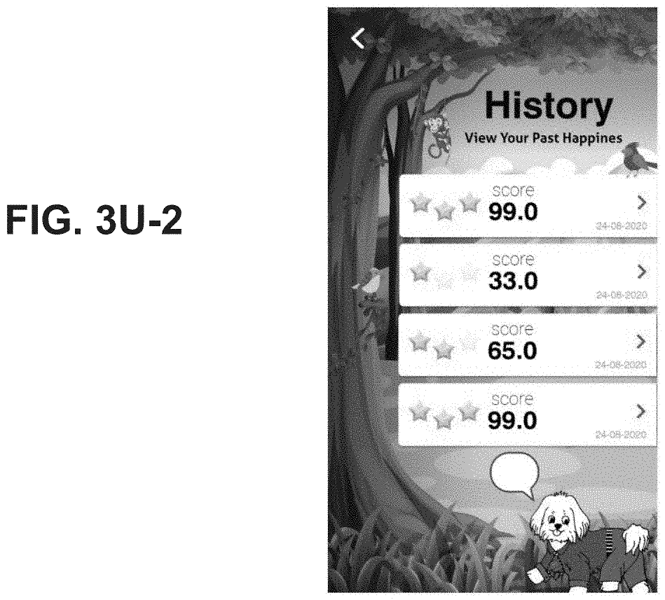

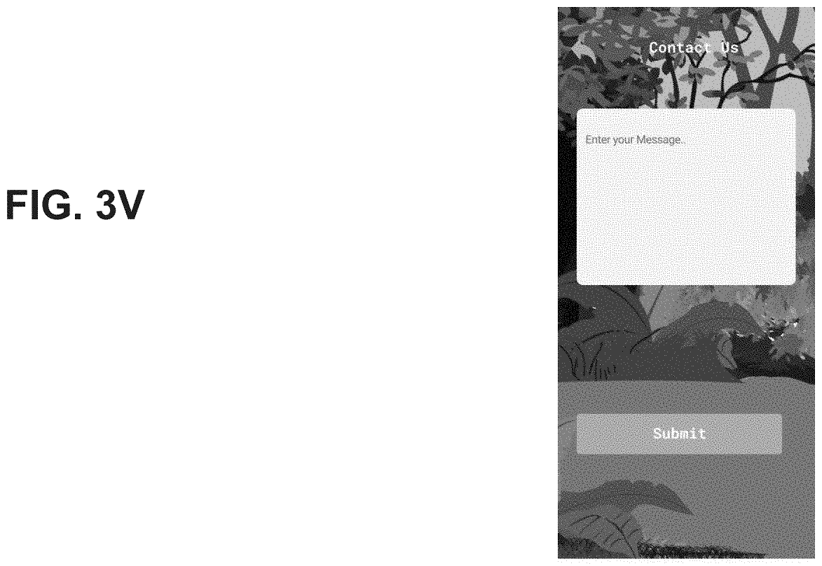

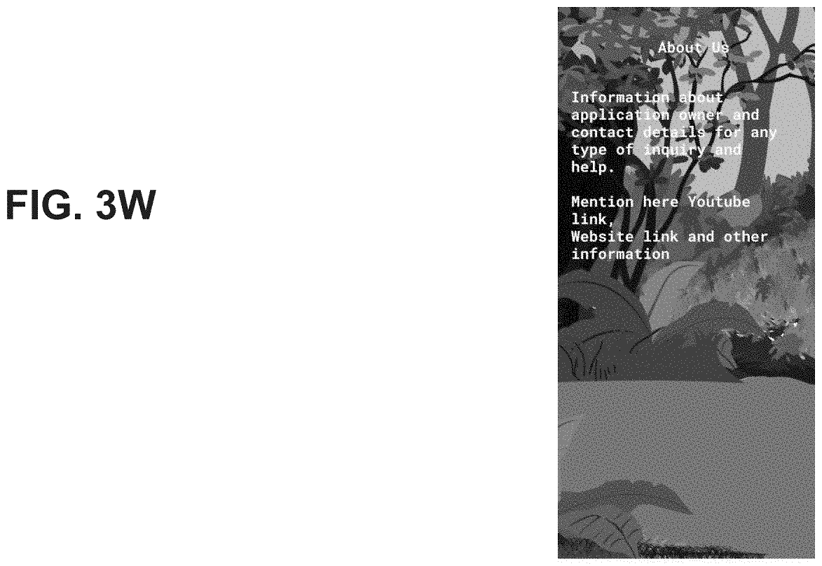

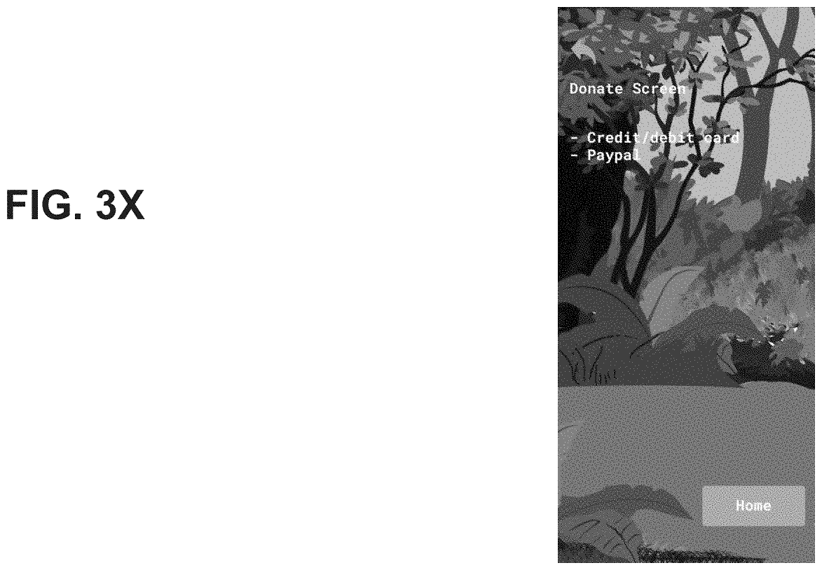

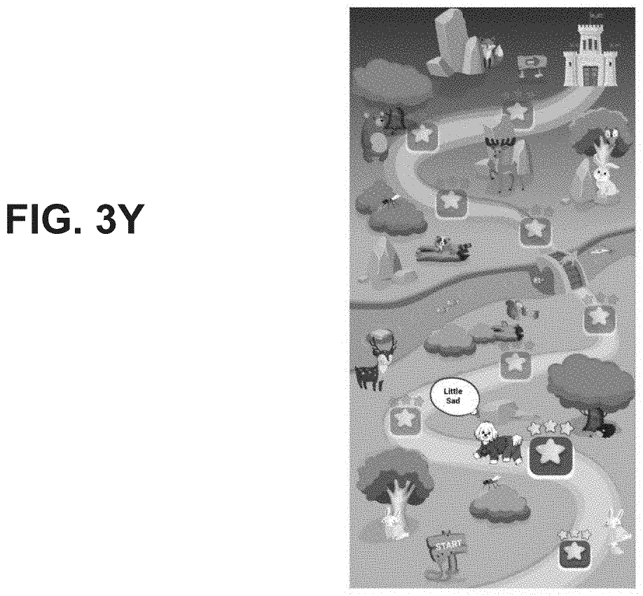

Embodiments may be understood with reference to the following sample screenshots of an example app, FIGS. 3A-3X. It should be appreciated that these screenshots are by way of example for illustrative purposes. FIG. 3A illustrates a cover screen; FIG. 3B illustrates a Login/Register/Social Login Screen; and FIG. 3C illustrates a Login/Register Screen. In an embodiment, if a user is new, then the system presents here a profile setup screen and obtains information by asking predetermined questions, such as a simple question about happiness. The profile screen is generic, meaning not having any of the happiness-related or solutions-related graphics. In an embodiment, the profile screen displays hobbies, etc. FIG. 3D illustrates a Jungle Puppy question screen. The purpose of the graphics in this screen are to begin to affect the person in a way to have the person begin their journey. In this example, the Jungle Puppy screen causes the user to feel happy. FIG. 3E illustrates the Happiness X land Game and provides text that explains the Happiness journey. FIG. 3F illustrates the dashboard with some data, such as History, Measure happiness, Donate, Contact Us, About Us, Profile, and an GUI for logout. FIG. 3G illustrates a Measure Happiness Expand in option and a Simple Question Screen: e.g., What's your definition of happiness? FIG. 3H illustrates a Measure Happiness Expand in option and on the screen asking What makes you happy? FIG. 3I illustrates a Measure Happiness Expand in option and a Simple Question Screen: What makes you unhappy? FIG. 3J illustrates a Measure Happiness Expand in option, the screen asking How do you rate yourself now? FIG. 3K illustrates a Measure Happiness Expand in option asking (a) How you rate your happiness now? (b) How do 3 of your closest peers rate you now? FIG. 3L-1 illustrates a Measure Happiness Expand in option, showing the Balloon Game and an Explanation of the Scores. In the example, the boxes are labeled Good and Bad. However, in an embodiment, they are labeled Happy and Unhappy. FIG. 3L-2 illustrates a loading screen that is displayed during the time that the happiness score is being loaded. It is specifically constructed to evoke a feeling of pleasantness of the user. FIG. 3M illustrates a Measure Happiness Expand in option, showing a Score Screen. While in the sample illustration, the score is shown as 9, in an embodiment, the score is typically between 1 and 3 in the beginning of the process. FIG. 3N-1 illustrates a Measure Happiness Expand in option, in which the Solution Screen displays a Video, Quiz, and Activity. In an embodiment, the data is used by the platform to generate and provide customized actions and solutions to the user. With the integration of artificial intelligence and predictive algorithms, the platform gains more data and thus clarity about the type of solutions will help the user achieve the desired emotional state. In an embodiment, the platform is configured to process virtual reality (VR) and augmented reality (AR) techniques to derive the customized, personalized, and relevant solutions for the individual. The variable solution is evident and displayed by the system when the user plays again after changing the score. FIG. 3N-2 illustrates a different solutions screen that displays links to a meditation video, to a game for playing, a quiz to interactively take, and other activities. FIG. 3O illustrates a Measure Happiness Expand in option, in which the Congratulation screen compares the user's score to scores of other players. FIG. 3P illustrates the Measure Happiness Expand in option, in which a rocket game is executed and displayed on the screen for the user. FIG. 3Q illustrates the Measure Happiness Expand in option, in which the user is shown the Congratulation screen, because the user's level of happiness was calculated to improve game. The screen depicts a pop up congratulating the user for improving their score. FIG. 3R illustrates a Measure Happiness Expand in option, by which the user can provide feedback and obtain more happiness. For example, the user can select Like or Unlike, Refer/Recommended and Collect Happy Karma points, Indicate what the user likes, doesn't like, and what changes they would like to see, and the system provide information about where the user can go for coaching sessions, seminars, webinars, obtain books, or go to a website for continued improvement. FIG. 3S illustrates a Measure Happiness Expand in option, from which the user can Donate or from which the user can Explore Happiness options in the present or correlated website. FIG. 3T illustrates a sample Dashboard, as mentioned above, in which the user can enter Profile information, such as name, email address, and phone number. FIG. 3U-1 illustrates a sample Dashboard showing the History tab information, such as old questions and answers, scores, and games played and FIG. 3U-1 shows a different example of a History of scores screen. FIG. 3V illustrates the sample Dashboard, when the Contact Us option has been selected. The user can enter a message and select the submit command to have the message sent to the appropriate, predetermined channel, such as a customer service representative. FIG. 3W illustrates a sample Dashboard, when the About Us option has been selected, Information about the application owner and contact details for types of inquiry and help are provided. Help videos, such as YouTube videos and the like, may be displayed, as well as links to the application owner's website or other helpful websites. FIG. 3X illustrates a sample Dashboard, when the Donate option has been selected. This screen is configured to obtain credit/debit card information, PayPal information, or other types of payment information. FIG. 3Y illustrates a wellness path screen, showing where along a wellness path the user is located, based on the user's happiness score. The goal at the end of the path is shown as a castle, to depict a high level of happiness or wellness.

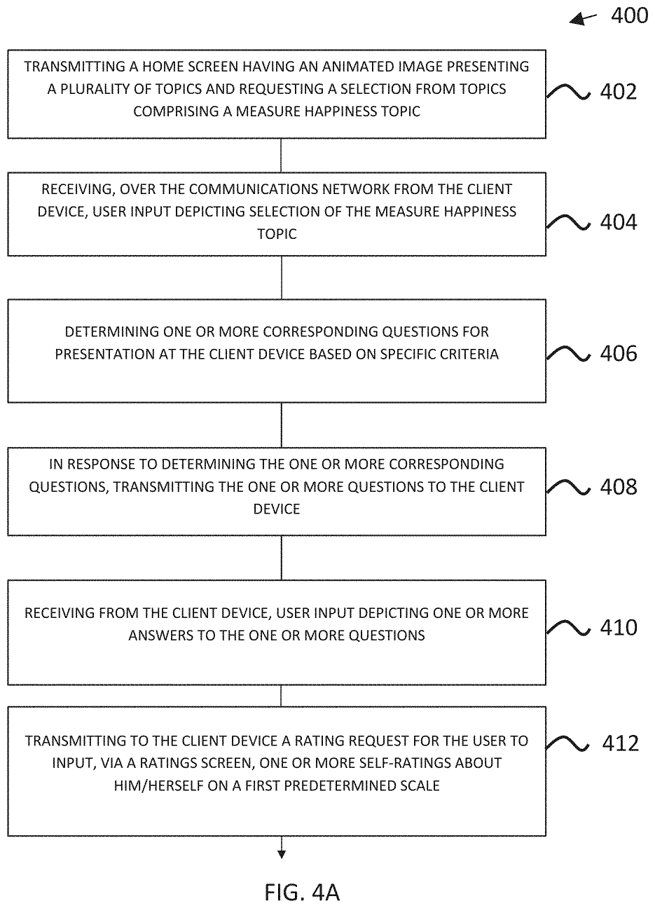

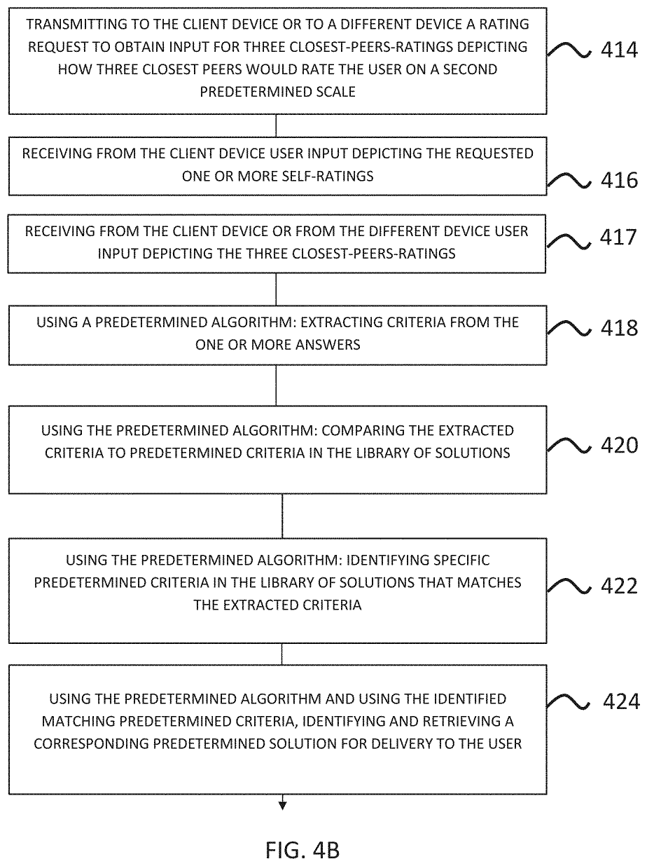

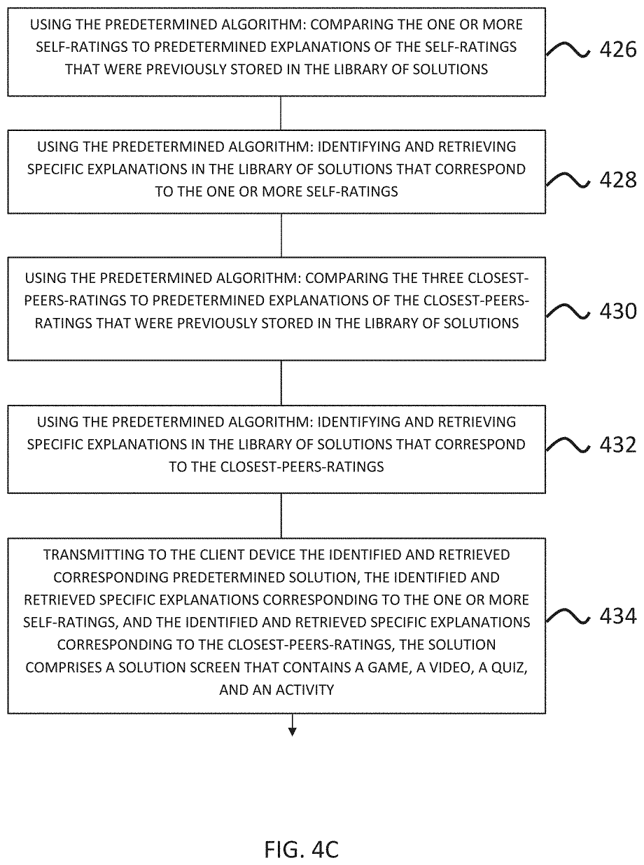

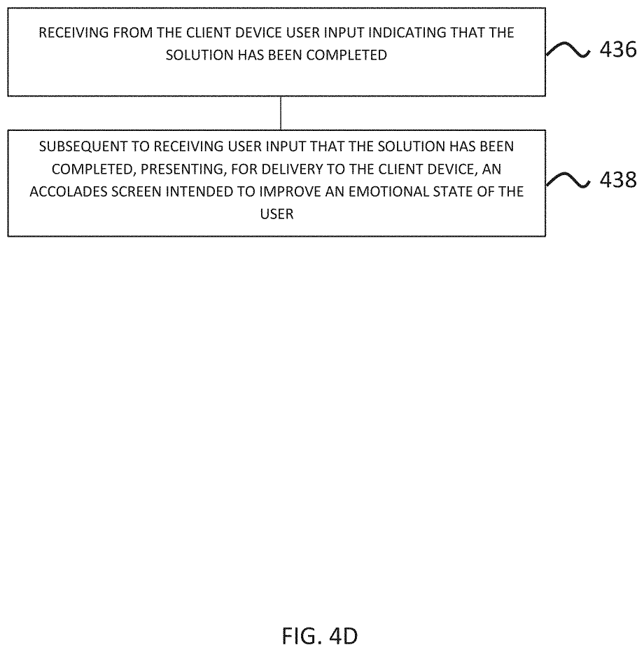

An embodiment can be understood with reference to FIG. 4, a flow diagram 400 showing an example of a method for generating a wellness solution in response to user input. At step 402, transmitting (e.g., server 202 and/or input/output processor 220), over a communications network (e.g., communications network 204) intended for a client device, a home screen having an animated image, wherein the animated image is configured to present (e.g., by animation processor and storage 216) a plurality of topics and requesting a selection from the plurality of topics, the plurality of topics comprising a measure happiness topic. At step 404, subsequent to transmitting the animated image, receiving (e.g., by input/output processor 220), over the communications network (e.g., communications network 204) from the client device, user input depicting selection of the measure happiness topic. At step 406, in response to receiving the user input depicting the selection of the measure happiness topic, determining (e.g., by processor for and library/repository of happiness questions 218) one or more corresponding questions for presentation at the client device based on specific criteria. At step 408, in response to determining the one or more corresponding questions, transmitting (e.g., by processor for and library/repository of happiness questions 218 and input/output processor and API with client device 220) the one or more questions to the client device. At step 410, receiving (e.g., by input/output processor and API with client device 220 and algorithm/processor for and library/repository of solutions and ratings/scores 222) from the client device, user input depicting one or more answers to the one or more questions. At step 412, transmitting (e.g., by input/output processor and API with client device 220 and algorithm/processor for and library/repository of solutions and ratings/scores 222) to the client device a rating request for the user to input, via a ratings screen (e.g., generated and provided by screen management analytics processor 214), one or more self-ratings about themselves on a first predetermined scale. At step 414, transmitting (e.g., by input/output processor and API with client device 220 and algorithm/processor for and library/repository of solutions and ratings/scores 222) to the client device or to a different device (not shown) a rating request to obtain input for three closest-peers-ratings depicting how three closest peers would rate the user on a second predetermined scale. At step 416, receiving (e.g., by input/output processor and API with client device 220) from the client device (e.g., by input/output processor and API with server 210 and GUI processor 208) user input depicting the requested one or more self-ratings. At step 417, receiving (e.g., by input/output processor and API with client device 220) from the client device (e.g., by input/output processor and API with server 210 and GUI processor 208) or from the different device (e.g., configured with an input/output processor and API with server similar to component 210 and GUI processor similar to component 208) user input depicting the three closest-peers-ratings. At step 418, using a predetermined algorithm (e.g., stored in algorithm/processor for and library/repository of solutions and ratings/scores 222), extracting criteria from the one or more answers. At step 420, using the predetermined algorithm (e.g., stored in algorithm/processor for and library/repository of solutions and ratings/scores 222), comparing the extracted criteria to predetermined criteria in the library of solutions. At step 422, using the predetermined algorithm (e.g., stored in algorithm/processor for and library/repository of solutions and ratings/scores 222), identifying specific predetermined criteria in the library of solutions that matches the extracted criteria. At step 424, using the predetermined algorithm (e.g., stored in algorithm/processor for and library/repository of solutions and ratings/scores 222) and using the identified matching predetermined criteria (e.g., stored in algorithm/processor for and library/repository of solutions and ratings/scores 222), identifying and retrieving a corresponding predetermined solution for delivery to the user. At step 426, using the predetermined algorithm (e.g., stored in algorithm/processor for and library/repository of solutions and ratings/scores 222) comparing the one or more self-ratings to predetermined explanations of the self-ratings that were previously stored in the library of solutions. At step 428, using the predetermined algorithm (e.g., stored in algorithm/processor for and library/repository of solutions and ratings/scores 222) identifying and retrieving specific explanations in the library of solutions that correspond to the one or more self-ratings. At step 430, using the predetermined algorithm (e.g., stored in algorithm/processor for and library/repository of solutions and ratings/scores 222) comparing the three closest-peers-ratings to predetermined explanations of the closest-peers-ratings that were previously stored in the library of solutions. At step 432, using the predetermined algorithm (e.g., stored in algorithm/processor for and library/repository of solutions and ratings/scores 222) identifying and retrieving specific explanations in the library of solutions that correspond to the closest-peers-ratings. At step 434, transmitting (e.g., by input/output processor and API with client device 220 and algorithm/processor for and library/repository of solutions and ratings/scores 222) to the client device (e.g., to input/output processor and API with server 210) the identified and retrieved corresponding predetermined solution, the identified and retrieved specific explanations corresponding to the one or more self-ratings, and the identified and retrieved specific explanations corresponding to the closest-peers-ratings. The solution comprises a solution screen that contains a game, a video, a quiz, and an activity (e.g., such screens generated and provided by screen management analytics processor 214 and intended for delivery to GUI processor 208). At step 436, receiving (e.g., by input/output processor and API with client device 220) from the client device (e.g., from input/output processor and API with server 210) user input indicating that the solution has been completed (e.g., by GUI processor 208). At step 438, subsequent to receiving user input that the solution has been completed, presenting (e.g., generated and provided by screen management analytics processor 214 and/or by animation processor and storage 216), for delivery to the client device (e.g., from input/output processor and API with client device 220, via communications network 204, to input/output processor and API with server 210) an accolades screen (e.g., generated and provided by screen management analytics processor 214 and/or by animation processor and storage 216) intended to improve an emotional state of the user.

### An Example Machine Overview

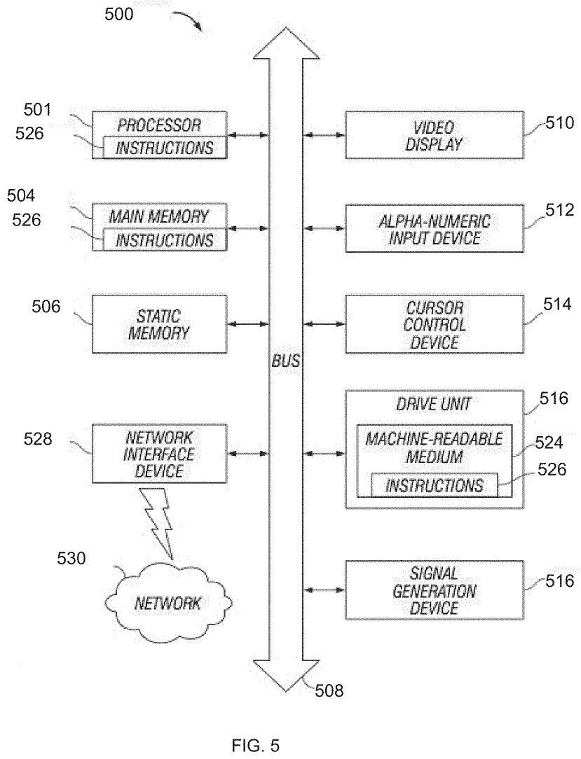

FIG. 5 is a block schematic diagram of a system in the exemplary form of a computer system 500 within which a set of instructions for causing the system to perform any one of the foregoing methodologies may be executed. In alternative embodiments, the system may comprise a network router, a network switch, a network bridge, personal digital assistant (PDA), a cellular telephone, a Web appliance or any system capable of executing a sequence of instructions that specify actions to be taken by that system.

The computer system 500 includes a processor 502, a main memory 504 and a static memory 506, which communicate with each other via a bus 508. The computer system 500 may further include a display unit 510, for example, a liquid crystal display (LCD) or a cathode ray tube (CRT). The computer system 500 also includes an alphanumeric input device 512, for example, a keyboard; a cursor control device 514, for example, a mouse; a disk drive unit 516, a signal generation device 518, for example, a speaker, and a network interface device 528.

The disk drive unit 516 includes a machine-readable medium 524 on which is stored a set of executable instructions, i.e. software, 526 embodying any one, or all, of the methodologies described herein below. The software 526 is also shown to reside, completely or at least partially, within the main memory 504 and/or within the processor 502. The software 526 may further be transmitted or received over a network 530 by means of a network interface device 528.

In contrast to the system 500 discussed above, a different embodiment uses logic circuitry instead of computer-executed instructions to implement processing entities. Depending upon the particular requirements of the application in the areas of speed, expense, tooling costs, and the like, this logic may be implemented by constructing an application-specific integrated circuit (ASIC) having thousands of tiny integrated transistors. Such an ASIC may be implemented with CMOS (complementary metal oxide semiconductor), TTL (transistor-transistor logic), VLSI (very large systems integration), or another suitable construction. Other alternatives include a digital signal processing chip (DSP), discrete circuitry (such as resistors, capacitors, diodes, inductors, and transistors), field programmable gate array (FPGA), programmable logic array (PLA), programmable logic device (PLD), and the like.

It is to be understood that embodiments may be used as or to support software programs or software modules executed upon some form of processing core (such as the CPU of a computer) or otherwise implemented or realized upon or within a system or computer readable medium. A machine-readable medium includes any mechanism for storing or transmitting information in a form readable by a machine, e.g. a computer. For example, a machine readable medium includes read-only memory (ROM); random access memory (RAM); magnetic disk storage media; optical storage media; flash memory devices; electrical, optical, acoustical or other form of propagated signals, for example, infrared signals, digital signals, etc.; or any other type of media suitable for storing or transmitting information.

Further, it is to be understood that embodiments may include performing operations and using storage with cloud computing and/or servers. For the purposes of discussion herein, cloud computing may mean executing algorithms on any network that is accessible by internet-enabled or network-enabled devices, servers, or clients and that do not require complex hardware configurations, e.g. requiring cables and complex software configurations, e.g. requiring a consultant to install. For example, embodiments may provide one or more cloud computing solutions that enable users, e.g. users on the go, to obtain wellness solutions on such internet-enabled or other network-enabled devices, servers, or clients. It further should be appreciated that one or more cloud computing embodiments include providing wellness solutions using mobile devices, tablets, and the like, as such devices are becoming standard consumer devices.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A method, comprising:
transmitting, over a communications network intended for a client device, a home screen having an animated image, wherein the animated image is configured to present a plurality of topics and requesting a selection from the plurality of topics, the plurality of topics comprising a measure happiness topic;
subsequent to transmitting the animated image, receiving, over the communications network from the client device, user input depicting selection of the measure happiness topic;
in response to receiving the user input depicting the selection of the measure happiness topic, determining one or more corresponding questions for presentation at the client device based on specific criteria;
in response to determining the one or more corresponding questions, transmitting the one or more questions to the client device;
receiving from the client device, user input depicting one or more answers to the one or more questions;
transmitting to the client device a rating request for the user to input, via a ratings screen, one or more self-ratings about him/herself on a first predetermined scale;
transmitting to the client device or to a different device a rating request to obtain input for three closest-peers-ratings depicting how three closest peers would rate the user on a second predetermined scale;
receiving from the client device user input depicting the requested one or more self-ratings;
receiving from the client device or from the different device user input depicting the three closest-peers-ratings;
using a predetermined algorithm:
extracting criteria from the one or more answers;
comparing the extracted criteria to predetermined criteria in the library of solutions;
identifying specific predetermined criteria in the library of solutions that matches the extracted criteria;
and using the identified matching predetermined criteria, identifying and retrieving a corresponding predetermined solution for delivery to the user;
comparing the one or more self-ratings to predetermined explanations of the self-ratings that were previously stored in the library of solutions;
identifying and retrieving specific explanations in the library of solutions that correspond to the one or more self-ratings;
comparing the three closest-peers-ratings to predetermined explanations of the closest-peers-ratings that were previously stored in the library of solutions; and
identifying and retrieving specific explanations in the library of solutions that correspond to the closest-peers-ratings;
transmitting to the client device the identified and retrieved corresponding predetermined solution, the identified and retrieved specific explanations corresponding to the one or more self-ratings, and the identified and retrieved specific explanations corresponding to the closest-peers-ratings;
wherein the solution comprises a solution screen that contains a game, a video, a quiz, and an activity;
receiving from the client device user input indicating that the solution has been completed; and
subsequent to receiving user input that the solution has been completed, presenting, for delivery to the client device, an accolades screen intended to improve an emotional state of the user.

2. The method of claim 1, further comprising:
providing for presentation at the client device, a screen or message box asking if the user would like to return to the solution screen to subsequently perform one of: improve score, retake the quiz, watch a video, engage in an activity, or engage in a game; and
returning control to the using a predetermined algorithm step until the user has stopped the process.

3. The method of claim 1, wherein the home screen, the rating screen, the solution screen, and the accolades screen are specifically designed with input from one or more wellness professionals to improve an emotional state of the user.

4. The method of claim 1, further comprising:
presenting a link to a preestablished website that provides wellness-related tools, services, and references to professionals, wherein the preestablished website is configured to accept user input indicative of user feedback intended for improving any of the process, the computation of the scores, the type of games, the type of videos, the type of quizzes, and the type of activities.

5. The method of claim 1, further comprising:
computing, at the server and transmitting to the client device, a percentile based on a current score of the user compared to scores of other anonymous users.

6. The method of claim 1, wherein an accolade includes a clapping animation.

7. The method of claim 1, wherein the game is any of: a balloon game and a rocket/parachute game; the video is a teaching/learning video related to wellness; the quiz is a Neuro Linguistic Programming (NLP) quiz; and the activity is any of: interactive, guided meditation, brief walk, dance, talk to a peer/friend, and correlates with the a desired outcome.

8. The method of claim 1, wherein the explanation of the score is derived from a matching component, wherein the matching component is prepopulated with a set of explanations and a set of ranges of scores, wherein each explanation from the set of explanations is matched with a specific range of the set of ranges.

9. The method of claim 1, wherein the predetermined algorithm is stored and executed on a cloud server and the ratings are pushed to the cloud server for processing by the predetermined algorithm.

10. The method of claim 1, wherein the predetermined algorithm is an averaging algorithm and wherein an initial user score is provided that is an average of the self-ratings and the closest-peers-ratings.

11. The method of claim 1, wherein the one or more self-ratings and the closest-peers-ratings are on a scale from 1 to 10.

12. The method of claim 1, wherein the one or more answers are obtained from a drop-down menu and/or from non-structured text in a text box.

13. The method of claim 1, wherein the one or more questions are configured to be stored in a temporary storage of the client device.

14. The method of claim 13, wherein the one or more questions are initially transmitted from a cloud server and are updates are automatically pushed from the cloud server when changes to the one or more questions are performed at the cloud server.

15. The method of claim 1, wherein the animated image is any of or any combination of audio and visual media.

16. The method of claim 1, wherein the client device is a desktop personal computer, smartphone, a laptop, and tablet.

17. A non-transitory digital storage medium having stored thereon a computer program for performing, when executed by a computer, a method for computing and presenting a wellness solution, the method comprising:
presenting, on a client device, a home screen with an animation, wherein the animation requests information about a topic;
subsequent to displaying the animation, receiving, at the client device, user input depicting a selection of the topic to measure happiness;
in response to receiving the user input selecting the topic to measure happiness, presenting, on the client device, one or more questions;
receiving, at the client device, user input depicting one or more answers to the one or more questions;
presenting, on the client device, a rating screen intended for the user to rate him/herself on a first predetermined scale and rate how a predetermined number of their closest peers would rate them on a second predetermined scale;
receiving, at the client device, user input depicting a first score representing how the user rates him/herself, and a second or more scores representing how the user believes each of his/her peers would rate the user;
using a predetermined algorithm, generating and presenting on the client device an initial user score and an explanation of the score;
in response to generating the initial user score, generating, based on the initial score, and presenting on the client device a solution screen, wherein the solution screen comprises a game, a video, a quiz, and an activity; and
receiving, at the client device, user input indicating that the user has completed the solution screen and, subsequently presenting an accolades screen intended to improve an emotional state of the user.

18. The non-transitory digital storage medium of claim 17, further comprising:
presenting, at the client device, a screen or message box asking if the user would like to return to the solution screen to subsequently retake the quiz and perform other actions to improve the initial score by processing data from the quiz, a prior score, and the game after the user retakes the game; and
returning control to the using a predetermined algorithm step until the user has stopped the process.

19. A system comprising:
a processor operable to execute computer program instructions; and
a memory operable to store computer program instructions executable by the processor, for performing:
presenting, on a client device, a home screen with an animation, wherein the animation requests information about a topic;
subsequent to displaying the animation, receiving, at the client device, user input depicting a selection of the topic to measure happiness;
in response to receiving the user input selecting the topic to measure happiness, presenting, on the client device, one or more questions;
receiving, at the client device, user input depicting one or more answers to the one or more questions;
presenting, on the client device, a rating screen intended for the user to rate him/herself on a first predetermined scale and rate how a predetermined number of their closest peers would rate them on a second predetermined scale;
receiving, at the client device, user input depicting a first score representing how the user rates him/herself, and a second or more scores representing how the user believes his/her peers would rate the user;
using a predetermined algorithm, generating and presenting on the client device an initial user score and an explanation of the score;
in response to generating the initial user score, generating, based on the initial score, and presenting on the client device a solution screen, wherein the solution screen comprises a game, a video, a quiz, and an activity; and
receiving, at the client device, user input indicating that the user has completed the solution screen and, subsequently presenting an accolades screen intended to improve an emotional state of the user.

20. The system of claim 19, wherein the processor and memory are each operable for further performing:
presenting, at the client device, a screen or message box asking if the user would like to return to the solution screen to subsequently retake the quiz and other actions to improve score; and
returning control to the using a predetermined algorithm step until the user has stopped the process.
